(19) United States
(12) Patent Application Publication
(10) Pub. No.: US 2006/0235214 A1
(43) Pub. Date: Oct. 19, 2006

(54) Identification of cancer genes using a high-throughput forward genetic screening method

(75) Inventor: David M. Morris, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/400,132

(22) Filed: Apr. 7, 2006

Related U.S. Application Data
(60) Provisional application No. 60/671,836, filed on Apr. 14, 2005.

(51) Int. Cl. C07H 21/04

(52) U.S. Cl. 536/23.2

(57) ABSTRACT

This invention relates to identification of cancer genes using a high-throughput forward genetic screening method called insertion mutation scanning (IMS).

### FIELD OF THE INVENTION

This invention relates to identification of cancer genes using a high-throughput forward genetic screening method called insertion mutation scanning (IMS).

### BRIEF SUMMARY OF THE INVENTION

The invention includes a highly efficient and scalable method for large scale isolation of host/provirus fragments. The invention is an anchored-PCR method designed to amplify host/virus junction fragments from new clonally integrated proviruses present in tumors induced by certain retroviruses or pathogenic endogenous proviruses. These proviruses represent the somatic mutations which caused the tumors from which the integrants were isolated. The IMS technique has utility with respect to a cancer gene discovery strategy called provirus tagging (see, e.g., US 2004/0142336A1). Because of the efficiency and scalability of the IMS procedure, it is useful technology that makes a saturation screen of the cancer genome feasible. The IMS procedure implements an anchored-PCR approach to provirus tagging. Advantages over previous methods include improvements in (1) sensitivity, (2) signal-to-noise ratio, (3) efficiency of mutation recovery, and (4) scalability. The invention includes nucleotide sequences (both complete sequences and host sequences) shown in Table 1.

### BRIEF DESCRIPTION OF THE DRAWINGS

FIG. 1 is a schematic of bioinformatics analysis.

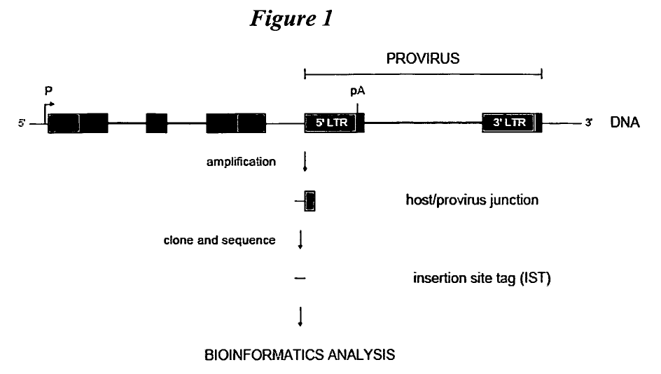

FIG. 2 shows the amplification results from 24 independent WXG/WLC tumors induced by MMTV. The upper common band contains the env region flanking the 3′ LTR and the lower common band contains the host sequence flanking the endogenous Mtv2 provirus which is common to all the tumors in the panel. Each unique band contains the 5′ host flanking sequence from a unique provirus insertion mutation that tags a cancer gene.

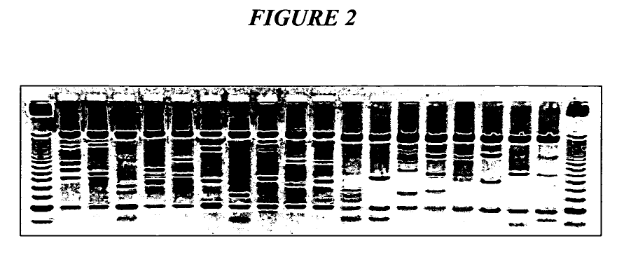

### DETAILED DESCRIPTION

The methods of the invention can be used to prepare nucleotide sequences from cancer genes. A sample protocol illustrating the methods of the invention is as follows.

1. Extract genomic DNA from frozen tumor tissues using a SNAP-O-SOL kit from Biotecx (Houston, Tex.), according to the manufacturer's instructions.

2. Digest 750 ng of genomic DNA with 10 units of NlaIII from New England BioLabs (Beverly, Mass.) in a total of 20 μl for one hour at 37° C. in the manufacturer's buffer (NEBuffer 4 with supplemental BSA).

3. Heat inactivate the enzyme at 65° C. for 25 min and place onto ice.

4. Ligate anchors onto 150 ng of NlaIII digested DNA (4 μl). Anchors consist of two annealed oligos with a 3′ CATG overhang. The oligos, synthesized by Integrated DNA Technologies (Coralville, Iowa), have the following sequence:
n0131 5′-CCTGA CTATG GAGTT CACTG CGTGC TACAC TATCA GTAGG CATG -3′
n0157 5′-CCTAC TGATA GTGTA GCACG-N-3′
note: n0157 contains a 3′ amino modifier (with a C7 spacer).
Prior to use, dilute oligos from a concentrated stock solution to 4 μM each in TE.
Mix equal parts to make a 2 μM solution.
Heat to 95° C. for 2 min and lower temperature to 45° C. for 10 min.

5. Ligation:

DNA Ligation Kit Version 1 from Takara Shuzo Co., Ltd. (Otsu; Shiga, Japan)
4 μl NlaIII digested DNA
1 μl 2 μM n0131/n0157
25 μl Solution A
05 μl Solution B
Mix all components on ice. Incubate at 16° C. for 1 hour.
Purify using Qiaquick PCR Purification Kit from Qiagen, Inc. (Santa Clara, Calif.) according to the manufacturer's instructions. Elute into 50 μl 10 mM Tris-HCl, pH 8.5 and place on ice.

6. Amplify the ligated material through two successive rounds of PCR using nested primers as follows.

7. PCR reaction 1:
5 μl anchor ligated DNA
1X Qiagen PCR Buffer with 1.5 mM MgCl2 (Qiagen; Santa Clara, Calif.)
200 μM dNTPs
50 U/ml HotStarTaq DNA Polymerase (Qiagen; Santa Clara, Calif.)
0.2 U Perfect Match PCR Enhancer (Stratagene; LaJolla, Calif.)
0.2 μM of each of the amplification primers
n0121 5′-CCTGA CTATG GAGTT CACTG-3′
n0138 5′-GATGG GCTCA TCCGT TTGTG-3′
H20 to a 50 μl reaction volume.
Perform the reactions in an Amplitron II thermal cycler from Thermolyne (Dubuque, Iowa) using a step down PCR reaction with denaturation, annealing, and extension temperatures of 94° C., 60-56° C., and 72° C., respectively. During the first 5 cycles, drop the annealing temperature in one degree increments from 60° C. to 56° C. Perform the last 60 cycles at a constant annealing temperature of 56° C., for a total of 65 cycles during the first round.
Cycling conditions:
95° C., 15 min dwell
2 cycles (94° C., 30 sec; 60° C., 30 sec; 72° C., 30 sec)
1 cycle (94° C., 30 sec; 59° C., 30 sec; 72° C., 30 sec)
1 cycle (94° C., 30 sec; 58° C., 30 sec; 72° C., 30 sec)
1 cycle (94° C., 30 sec; 57° C., 30 sec; 72° C., 30 sec)
60 cycles (94° C., 30 sec; 56° C., 30 sec; 72° C., 30 sec)
72° C., 10 min dwell

8. PCR reaction 2:

9. Shotgun clone 1 μl of this PCR product using a TA TOPO Cloning Kit from Invitrogen (Carlsbad, Calif.), according to the manufacturer's instructions with the following exception. Do not incubated transformed cells in a shaker after heat shock and subsequent ice step. Plate directly onto prewarmed LB Amp100/Kan 5 plates to ensure more accurate cloning representation.

10. PCR screen white colonies for insert size as described below.

11. Grown all screened colonies overnight in 2 ml LB Amp100/Kan50.

12. Miniprep cultures with correct insert sized using a QIAprep Miniprep Kit from Qiagen (Santa Clara, Calif.).

13. Send plasmids to University of California, Davis DBS Automated Sequencing Facility for insert sequence determination using PRISM sequencers, reagents, and software from ABI (Foster City, Calif.).

14. PCR screening for insert size:
Pick 24 white colonies/tumor into 20 μl LB Amp100/Kan50.
PCR reaction:
1 μl culture
1X Qiagen PCR Buffer with 1.5 mM MgCl2 (Santa Clara, Calif.)
200 μM dNTPs
20 U/ml Qiagen Taq Polymerase
0.2 μM each of the amplification primers
n0122 5′-CGTGC TACAC TATCA GTAGG-3′
n0036 5′-CAACC CCTTG GCTGC TTCTC-3′
50 μl reaction volume.

Perform the reactions in an Amplitron II thermal cycler from Thermolyne (Dubuque, Iowa) for 30 cycles with denaturation, annealing, and extension temperatures of 94° C., 56° C., and 72° C., respectively.

TABLE 1
IST Sequences

| | | | | | |
|---|---|---|---|---|---|
| | | | | INSERT | CANCER |
| TAG | BAND | COMPLETE SEQUENCE | HOST SEQUENCE | LENGTH | GENE |

| | | | | | |
|---|---|---|---|---|---|
| IST000001 | MM0001B | GAATTCGCCCTTCGTGCTACACTATCAGTAGGCAT | CATGAAGACCCCAGGCTCAGTT | 264 | unknown |
| | | GAAGACCCCAGGCTCAGTTTCTGCTCCCAGGAGAA | TCTGCTCCCAGGAGAAAGTCCT | | |
| | | AGTCCTGCTCTCTAGTCTGCCCTGAGGGCCAAACA | GCTCTCTAGTCTGCCCTGAGGG | | |
| | | CGGGACTCTTGGTGCACCTCGTCAGTTTTGTGCAG | CCAAACACGGGACTCTTGGTGC | | |
| | | TGCTTTAGCAGGGCCAGAGAGCCCATCTAGTGGCC | ACCTCGTCCAGTTTTGTGCAGT | | |
| | | AGGGCAGAACCCTGTCCCTGGCTGCATCTGGTCTT | GCTTTAGCAGGGCCAGAAGCCC | | |
| | | CAGCTCAGGAATGTGAGATTGTCTTGTTAGTTTTT | ATCTAGTGGCCAGGGCAGAACC | | |
| | | GAAGTAACACAGTTCCTGCCTAGCTCCAAGAGTGG | CTGTCCCTGGCTGCATCTGGTC | | |
| | | CGCTCCCTCCCACTTGCCGCGCCTGCAGCAGAAAT | TTCAGCTCAGGAATGTGAGATT | | |
| | | GGTTGAACTCCCGAGAGTGTCCTACACTTAGGGGA | GTCTTGTTAGTTTTTGAAGTAA | | |
| | | GAAGCAGCCAAGGGGTTGAAGGGCGAATTC | CAACAGTTCCTGCCTAGCTCCA | | |
| | | | AGAGTGGCGCTCCTCCCACT\hat{ } | | |
| IST000002 | MM0001C | GAATTCGCCCTTCGTGCTACACTATCAGTAGGCAT | CATGCTCATAATTCTGCAGTGC | 156 | unknown |
| | | GCTCATAATTCTGCAGTGCCTTCTCATAACACAGG | CTTCTCATAACACAGGATAAAA | | |
| | | ATAAAACACTCTAACCTTTAACATTATACTTGAAA | CACTCTAACCTTTAACATTATA | | |
| | | ACTTATGTGGTTTTTTCCTACCGAGTCATATCAAA | CTTGAAAACTTATGTGGTTTTT | | |
| | | CCAGTCTCCCTCTCCACTCACAAGGATCCAGTCAC | TCCTACCAGAGTCATATCAAAC | | |
| | | AATGGCCTTTTATGCCGCGCCTGCAGCAGAAATGG | CAGTCTCCCTCTCCACTACAAG | | |
| | | TTGAACTCCCGAGAGTGTCCTACACTTAGGGGAGA | GATCCAGTCACAATGGCCTTTT | | |
| | | AGCAGCCAAGGGTTGAAGGGCGAATTC | A\hat{ } | | |
| IST000003 | MM0001D | GAATTCGCCCTCGTGCTACACTATCAGTAGGCAT | CATGTCCTACAGTGAACATTTC | 130 | unknown |
| | | GTCCTACAGTGAACATTTCTATATTTTCCACTTTT | TATATTTTCCACTTTTTTCAGT | | |
| | | TTCAGTTTTCCTCGCCATATTTCACGTCCTAAAGT | TTTCCTCGCCATATTTCACGTC | | |
| | | GTGTATTTCTCATTTTCCGTGATTTCAGTTTTCTC | CTAAAGTGTGTATTTCTCATTT | | |
| | | GCCATATTCCAGGTCCTACAGTGCCGCGCCTGCAG | TCCGTGATTTTCAGTTTTCTCG | | |
| | | CAGAAATGGTTGAACTCCCGAGAGTGTCCTACACT | CCATATTCCAGGTCCTAAG\hat{ } | | |
| | | TAGGGGAGAAGCAGCCAAGGGGTTGAAGGGCGAAT | | | |
| | | TC | | | |
| IST000004 | MM0001E | GAATTCGCCCTTCGTGCTACACTATCAGTAGGCAT | CATGGCGAGATTCTGTGTCCAA | 101 | Wnt3 |
| | | GGCGAGATTCTGTGTCCAAGCTGCCTCTACTCGTG | GCTGCCTCTACTCGTGACATTC | | |
| | | ACATTCCAAGATGCCTCTGAGGTGGGAACTGTGAA | CAAGATGCCTCTGAGGTGGGAA | | |
| | | ATAGGAGAGAGCCCCACAGTCCCTCTTTGCCGCGC | CTGTGAAATAGGACAGAGCCCC | | |
| | | CTACAACAAAAATGGTTGAACTCCCGAGAGTGTCC | ACAGTCCCCTCTT\hat{ } | | |
| | | TACACTTAGGGGAGAAGCAGCCAAGGGGTTGAAGG | | | |
| | | GCGAATTC | | | |
| IST000005 | MM0007C | GAATTCGCCCTTCGTGCTACACTATCAGTAGGCAT | CATGTGTTTCCCGATTTTAGTT | 89 | unknown |
| | | GTGTTTCCCGATTTTAGTTGGATTTTTTTTCTCCC | GGATTTTTTTTCTCCCAGGTTT | | |
| | | AGGTTTCTGCAGTGTCCCCACCCCCCACCCCCATT | CTGCAGTGTCCCCACCCCCCAC | | |
| | | TTCTCCCACTCTTACCTGCCGCCCTGCAGCAGAAA | CCCCATTTTCTCCCACTCTTAC | | |
| | | TGGTTGAACTCCCGAGAGTGTCCTACACTTAGGGG | C\hat{ } | | |
| | | AGAAGCAGCCAAGGGGTTGAAGGGCGAATTC | | | |
| IST000006 | MM0014B | GAATTCGCCCTTCGTGCTACACTATCAGTAGGCAT | CATGGTGACAGTCCCTGAGTAA | 106 | unknown |
| | | GGTGACAGTCCCTGAGTAAAGGCTGGCTACTCCAT | AGGCTGGCTACTCCATCTTAGT | | |
| | | CTTAGTGGATTTGTTTTTATAAAGAAGGCAGACGA | GGATTTGTTTTTATAAAGAAGG | | |
| | | GAACCAGAAATAACAACACATTTAAGCGTATGTGC | CAGACGAGAACCAGAAATAACA | | |
| | | CGCGCCTGCAGCAGAAATGGTTGAACTCCCGAGAG | ACACATTGTAAGCGTATG\hat{ } | | |
| | | TGTCCTACACTTAGGGGAGAAGCAGCCAAGGGGTT | | | |
| | | GAAGGGCGAATTC | | | |
| IST000007 | MM0008B | GAATTCGCCCTTCGTGCTACACTATCAGTAGGCAT | CATGTATTTTTGCTGGGACTGT | 50 | unknown |
| | | GTATTTTTGCTGGGACTGTTCCCTCTGGGAACTGC | TCCCTCTGGGAACTGCGGGATG | | |
| | | GGGATGGGGGTGTGCCGCGCCTGCAGCAGAAATGG | GGGGTG\hat{ } | | |
| | | TTGAACTCCCGAGAGTGTCCTAACTTAGGGGAGAA | | | |
| | | GCAGCCAAGGGGTTGAAGGGCGAATTC | | | |
| IST000008 | MM0003B | GAATTCGCCCTTCGTGCTACACTATCAGTAGGCATG | CATGGCAAGATGGAGACTTTG | 75 | Fgf3 |
| | | GCAAGATGGAGACTTTGTCTACCAGGGCCACTCCAA | TCTACCAGGGCCACTCCAAGC | | |
| | | GCACCCAGCTGCATACAGGTGGACTGGCTGTGGCCT | ACCCAGCTGCATACAGGTGGA | | |
| | | GCCGCGCCTGCAGCAGAAAGGTTGAACTCCCGAGAG | CTGGCTGTGGCC\hat{ } | | |
| | | TGTCCTACACTTAGGGGAGAAGCAGCCAAGGGGTTG | | | |
| | | AAGGGCGAATTC | | | |
| IST000009 | MM0010B | GAATTCGCCCTTCGTGCTACACTATCAGTAGGCATG | CATGTAAGAAAAACAAGATCA | 170 | unknown |
| | | TAAGAAAAACAAGATCAAAGAAATGAGTCAAAAAGG | AAGAAATGAGTCAAAAAGGCT | | |
| | | CTCTAACAGGGCTTGAAGGTGAGGTAGAAATGACAG | CTAACAGGGCTTGAAGGTGAG | | |
| | | AAGACATAGAAATCCAATATCAGAAGGCAGAGTTTG | GTAGAAATGACAGAAGACATA | | |
| | | TATGGGACAGATACTCTCTATATATATGCTAGCACA | GAAATCCAATATTCAGAAGGC | | |
| | | CCTACTACTCAGCTGTTTCTCTGCCGCGCCTGCAGC | AGAGTTTGTATGGGACAGATA | | |
| | | AGAAATGGTTGAACTCCCAAGAGTGTTCTACACTTA | CCTCTATATATATGCTAGCAC | | |
| | | GGGAGAAGCAGCCAAGGGGTTGAAGGGCGAATTC | ACCTACTACTCAGCTGTTTCT | | |
| | | | C\hat{ } | | |
| IST000010 | MM0015B | GAATTCGCCCTTCGTGCTACACTATCAGTAGGCATG | CATGTCCTACAGTGGATATTT | 135 | unknown |
| | | TCCTACAGTGGATATTTCTAAATTTTCCTCCTTTTT | CTAAATTTTCCTCCTTTTTCA | | |
| | | CAGTTTTCCTCGCCATATTTGAAGTCCTAAAGTGTG | GTTTTCCTCGCCATATTTGAA | | |
| | | TATTTCTCATATCCTGTGATTTCAGTTTTCTCSCCA | GTCCTAAAGTGTGTATTTCTC | | |
| | | TATTCCAGGTCCTACAGTGTGCTGCCGC0CCTGCAG | ATATCCTGTGATTTTCAGTTT | | |
| | | CAGAAATGGTTGAACTCCCGAGAGTGTCCTACACTT | TCTCGCCATATTCCAGGTCCT | | |
| | | AGGGGAGAAGCAGCCAAGGGGTCGAAGGGCGAATTC | AAGTGTGC\hat{ } | | |
| IST000011 | MM0015C | GAATTCGCCCTTCGTGCTACACTATCAGTAGGCATG | CATGTGGAGGCCAGAAGTCAA | 59 | unknown |
| | | TGGAGGCCAGAAGTCAACATATAGTCTCCTTCCCAA | CATATAGTCTCCTTCCCAATT | | |
| | | TTACTTGTCACTGGAGAGCTGCCGCGCCTGCAGCAG | ACTTGTCACTGGAGAGC\hat{ } | | |
| | | AAATGGTTGAACTCCCGAGGTGTCCTACACTTAGGG | | | |
| | | GAGAAGCAGCCAAGGGGTTGAAGGGCGAATTC | | | |
| IST000012 | MM0013B | GAATTCGCCCTTCGTGCTACACTATCAGTAGGCATG | CATGCTACATTTTCATATTGT | 56 | unknown |
| | | CTACATTTTCATATTGTGTGCATACAAGGTTTCCCT | GTGCATACAAGGTTTCCCTTT | | |
| | | TTATTATGTGGGGTAATGCCGCGCCTGCAGCAGAAA | ATTATGTGGGGTAA\hat{ } | | |
| | | TGGTTGAACTCCCGAGAGTTCCTACACTTAGGGGAG | | | |
| | | AAGCAGCCAAGGGGTTGAAGGGCGAATTC | | | |
| IST000013 | MM0013B | GAATTCGCCCTTCGTGCTACACTATCAGTAGGAATG | AATGGGAGGAAGGACTGTGGA | 43 | unknown |
| | | GGAGGAAGGACTGTGGAAGGCAGAGGTGGAAATATC | AGGCAGAGGTGGAAATATCTG | | |
| | | TGCTGCCGCGCCTGCAGCAGAAATGGTTGAACTCCC | C\hat{ } | | |
| | | GAGAGTGTCCTACACTTAGGGAGAAGCAGCCAAGGG | | | |
| | | GTTGAAGGGCGAATTC | | | |
| IST000014 | MM0006B | GAATTCGCCCTTCGTGCTACACTATCAGTAGGCATG | CATGCTGGCTGTTCCTGCAGC | 46 | Fgf8 |
| | | CTGGCTGTTCCTGCAGCCCAGCTACTAGGACAATCT | CCAGCTACTAGGACAATCTGG | | |
| | | GGAAACTGCCGCGCCTGCAGCAGAAATGGTTGAACT | AAAC\hat{ } | | |
| | | CCCGAGAGTGTCCTACACTAGGGGAGAAGCAGCCAA | | | |
| | | GGGGTTGAAGGGCGAATTC | | | |
| IST000015 | MM0007B | GAATTCGCCCTTCGTGCTACACTATCAGTAGGCATG | CATGGAAAGATAATGTGTAAA | 244 | unknown |
| | | GAAAGATAATGTGTAAATTTGGGTTTGCCGTGGAAA | TTTGGGTTTGCCGTGGAAAAC | | |
| | | ACTTTGGTTTCTCCATCAATGGTAATTGAGAGTTTG | TTTGGTTTCTCCATCAATGGT | | |
| | | GCTGGGTATAGTAGCCTGGCTGGCATTTTTGTTCTC | AATTGAGAGTTTGGCTGGGTA | | |
| | | TTAAGGTCTGTATAAAGTCTGTCCAGGATCTTCTGA | TAGTAGCCTGGGCTGGCATTT | | |
| | | CTTTCATAATGTCTGGTGTAAAGTCTGGTGTAATTC | TTGTTCTCTTAAGGTCTGTAT | | |
| | | TGACAGGCCTGCCTTTATATGTTACTTGACCTTTTT | AAGTCTGTCCAGGATCTTCTG | | |
| | | CCTTACTGCTTTTAATATTCTATGCCGCGCCTGCAG | ACTTTCATAATGTCTGGTGTA | | |
| | | CAGAAATGGTTGAACTCCCGAGAGTGTCCTACACTT | AAGTCTGGTGTAATTCTGACA | | |
| | | AGGGGAGAAGCAGCCAAGGGGTTGAAGGGCGAATTC | GGCCTGCCTTTATATGTTACT | | |
| | | | TGACCTTTTTCCCTTACTGCT | | |
| | | | TTTAATATTCTA\hat{ } | | |
| IST000016 | MM0010C | GAATTCGCCCTTCGTGCTACACTATCAGTAGGCATG | CATGGGTCCCTGAAGGGTCTC | 79 | unknown |
| | | GGTCCCTGAAGGGTCTCTCCTTTAGCAAACCCCTGT | TCCTTTAGCAAACCCCTGTAC | | |
| | | ACAGTTGAAGTGATTTTTCAGGTACCCATTGGTCTT | AGTTGAAGTGATTTTTCAGGT | | |
| | | AGCTGCCGCGCCTGCAGCAAAATGGTTGAACTCCCG | ACCCATTGGTCTTAGC\hat{ } | | |
| | | AGAGTGTCCTACACTTAGGGGAGAAGCAGCCAAGGG | | | |
| | | GTTGAAGGGCGAATTC | | | |
| IST000017 | MM0004B | GAATTCGCCCTTCGTGCTACACTATCAGTAGGCA | CATGTTGTCCTTTGAGCATCTC | 127 | unknown |
| | | TGTTGTCCTTTGAGCATCTCCACCTAGCAGCTGA | CACCTAGCAGCTGACTTAGACA | | |
| | | CTTAGACAGATACACACACCCACAGGTGAACAGT | GATACACACACCCACAGGTGAA | | |
| | | GGATGGAGATTGGGGACTCTTATGGAGAATGGGA | CAGTGGATGGAGATTGGGGACT | | |
| | | GGAAGGATTTTGGGCCCTAAGGTGCCGCGCCTGC | CTTATGGAAGAATGGGAGGAAG | | |
| | | AGCAGAAATGGTTGAACTCCCGAGAGTGTCCTAC | GATTTTGGGCCCTAAGG\hat{ } | | |
| | | ACTTANGGGAGAAGCAACCAAGGGGTGAAGGGCG | | | |
| | | AATTC | | | |
| IST000018 | MM0004C | GAATTCGCCCTTCGTGCTACACTATCAGTAGGCA | CATGTGACACTGGCTTTGGAGT | 81 | unknown |
| | | TGTGACACTGGCTTTGGAGTCATACATAGAAGGG | CATACATAGAAGGGGTTGGGAC | | |
| | | GTTGGGACAATTGATGCTAGTTAGCTAGAGCTAA | AATTGATGCTAGTTAGCTAGAG | | |
| | | GAATATAGCAGTGCCGCGCCTGCAGAGAAATGGT | CTAAGAATATAGCAG\hat{ } | | |
| | | TGAACTCCCGAGAGTGTCCTACACTTAGGGGAGA | | | |
| | | AGCAGCCAAGGGGTTTAAGGGCGAATTC | | | |
| IST000019 | MM0005B | GAATTCGCCCTTCGTGCTACACTATCAGTAGGCA | CATGGTCTCTAGAGCTAAGAGA | 173 | unknown |
| | | TGGTCTCTAGAGCTAAGAGATACCAATGCTGCGG | TACCAATGCTGCGGCAGGCAGT | | |
| | | CAGGCAGTTTTTATTACAATCATTACAGTTTTGA | TTTTATTACAATCATTACAGTT | | |
| | | CAGCGTCTGGCCGTGTGCCAAGGCTGCCTTCATC | TTGACAGCGTCTGGCCGTGTGC | | |
| | | CCTGAGCTCGGTGATGCTTCTGTCCTGGTCTTCT | CAAGGCTGGCCTTCATCCCTGA | | |
| | | GGCTCATCACAGCTTAAGAAAGTAGCTGCTTCTC | GCTCGGTGATGCTTCTGCCTGG | | |
| | | TGCCGCGCCTGCAGCAGAAATGGTTGAACTCCCG | TCTTCTGGCTCATCACAGCTTA | | |
| | | AGAGTGTCCTACACTTGGGGAGAAGCAGCCAAGG | AGAAAGTAGCTGCTTCTC\hat{ } | | |
| | | GGTTGAAGGGCGAATTC | | | |
| IST000020 | MM0002C | GAATTCGCCCTTCGTGCTACACTATCAGTAGGGA | GATGGAACACTCAAAGCTGGCC | 115 | unknown |
| | | TGGAACACTCAAAGCTGGCCAGGGCCCATTTACC | AGGGCCCATTTACCAGGTATCC | | |
| | | AGGTATCCTTTGCCTTCTCAGCTGATGGGCATCA | TTTGCCTTCTCAGCTGATGGGC | | |
| | | ACACATTAATTCACATATGACTCGTTGTGTCATA | ATCAACACATTAATTCACATAT | | |
| | | TCAATAGTATTGCCGCGCCTGCAGCAGAAATGGT | GACTCGTTTGTGTCATATCAAT | | |
| | | TGAACTCCCGAGAGTGTCCTACACTTAGGGGAGA | AGTAT\hat{ } | | |
| | | AGCAGCCAAGGGGTTGTTTCCCACCAAGGACGAC | | | |
| | | CCGTCTGCGCACAAACGATGAGCCCATCAAGGGC | | | |
| | | GAATTC | | | |
| IST000021 | MM0002B | GAATTCGCCCTTCGTGCTACACTATCAGTAGGCA | CATGGGGGGGGGTACCAAGAAG | 271 | unknown |
| | | TGGGGGGGGGTACCCAAGAAGGGACTGCTGCGAT | GGACTGCTGCGATTGGGATGTA | | |
| | | TGGGATGTAAATAAATAAATAAATAGAATAAACA | AATAAATAAATAAATAGAATAA | | |
| | | AAACCCAAAAACAAACAGAAACCTAACTCAATAA | ACAAAACCCAAAAACAAACAGA | | |
| | | CTGCAGAAATGACTCTTGCTCTTTTCTGGTAAGG | AACCTAAACTCAATAACTGCAG | | |
| | | TTAGAAGCAGGTTACAAATCTATATTAGAGATGG | AAATGACTCTTGCTCTTTCTGG | | |
| | | AGGCATTTCACACCAGCATAGGTATAGGAAGTAG | TAAGGTTAGAAGCAGGTTACAA | | |
| | | ATGAAATGAGGACTACCTAGAGTCTGTTTGTCAC | ATCTATATTAGAGATGGAGGCA | | |
| | | AACCAATTCTGAGTGATTTCACTGAGATATTGCC | TTTCACACCAGCATAGGTATAG | | |
| | | GCGCCTGCAGCAGAAATGGTTGAACTCCCGAGAG | GAAGTAGATGAAATGAGGACTA | | |
| | | TGTCCTACACTTAGGGGAGAAGCAGCCAAGGGGT | CACTAGAGTCTGTTTGTCACAA | | |
| | | TGAAGGGGAATTC | CCAATTCTGAGTATTTCACTGA | | |
| | | | GATAT\hat{ } | | |
| IST000022 | MM0016D | GAATTCGCCCTTCGTGCTACACTATCAGTAGGGA | GATGTACTGAATCCCTGAAGTT | 66 | unknown |
| | | TGTACTGAATCCCTGAAGTTGATGCTGAGCACCA | GATGCTGAGCACCATCTTGTGC | | |
| | | TCTTGTGCTGTTCTACCGCATTTACTGGGGTGCC | TGTTCTACCGCATTTACTGGGG\hat{ } | | |
| | | GCGCCTGCAGCAGAAATGGTTGAACCCCGAGAGT | | | |
| | | GTCCTACACTTAGGGGAGAAGCAGCCAAGGGGTT | | | |
| | | GAAGGGCGAATTC | | | |
| IST000023 | MM0016B | GAATTCGCCCTTCGTGCTACACTATCAGTAGGCA | CATGAGTACATTTCATTTTTTC | 212 | unknown |
| | | TGAGTACATTTCATTTTTTCCCACAAACTGCACA | CCACAAACTGCACAGACCAACT | | |
| | | GACCAACTTTGAGAAATGCCTGTTTATGAATGAC | TTGAGAAATGCCTGTTTATGAA | | |
| | | CAGAGATCACGTCTTCCATTTTTAACACTTAGAG | TGACCAGAGATCACGTCTTCCA | | |
| | | ATGTGGAAGACTTCTGGTTAGCCCACTTATCCCT | TTTTTAATCACTTAGAGATGTG | | |
| | | TCCCAGACTAGCAGTCAAGGCCAAGCAAAGGGAG | GAAGACTTCTGGTTAGCCACTT | | |
| | | AATTTGGGCTGGTCCTATCCCCCCTGCTGGCTAC | ATCCCTTCCCAGACTAGCAGTC | | |
| | | TGAACTGCCGCGCCTGAGCAGAAATGGTTGAACT | AAGGCCAAGCAAAGGGAGAATT | | |
| | | CCCGAGGGTGTCCTACACTTAGGGGAGAAGCAGC | TGGGCTGGTCCTATCCCCCCTG | | |
| | | CAAGGGTTGTTTCCCACCAAGGACGACCCGTCTG | CTGGCTACTGAAC\hat{ } | | |
| | | CGCACAAACGGATGAGCCCATCAAGGGCGAATTC | | | |
| IST000024 | MM0016C | GAATTCGCCCTTCGTGCTACACTATCAGTAGG | CATGGATTATCTCTCTTCTATCCTT | 125 | unknown |
| | | CATGGATTATCTCTCTTCTATCCTTCCATTTT | CCATTTTTGTTTTTGTTTGTTGGGT | | |
| | | TGTTTTTGTTTGTTGGGTTGGTTTTGATTTTT | TGGTTTTGATTTTTGAGGCAGGGTT | | |
| | | GAGGCAGGGTTTCCCTGTGCATCCCTGCCTGC | TCCCTGTGCATCCCTGCCTGGCTAT | | |
| | | TATCCTCGAACTTGACCTCACTCTGTAGCGCC | CCTCGAACTTGACCTCACTCTGTAG\hat{ } | | |
| | | GCGCCTGCAGCAGAAATGGTTGAACTCCCGAG | | | |
| | | AGTGTCCTACACTTAGGGGAGAAGCAGCCAAG | | | |
| | | GGGTTGAAGGGCGAATTC | | | |
| IST000025 | MM0009C | GAATTCGCCCTTCGTGCTACACTATCAGTAGG | CATGTAATGCACAATATATGAGTCC | 96 | unknown |
| | | CATGTAATGCACAATATATGAGTCCTGATATT | TGATATTTATCCCTTACAGTCAATA | | |
| | | TATCCCTTACAGTCAATATAGCTTTGTCTACA | TAGCTTTGTCTACAAATTTTTGACA | | |
| | | AATTTTTGACATAAAGTAGGGCTATTTTTCAT | TAAAGTAGGGCTATTTTTCAA\hat{ } | | |
| | | GCCGCGCCTGCAGCAGAAATGGTTGAACTCCC | | | |
| | | GAGAGTGTCCTACACTTAGGGGAGAAGCAGCC | | | |
| | | AAGGGGTTGAAGGGCGAATTC | | | |
| IST000026 | MM0009B | GAATTCGCCCTTCGTGCTACACTATCAGTAGG | CATGGAAATGCAATGAAAGCACACA | 301 | Unknown |
| | | CATGGAAATGCAATGAAAGCACACAATATTGT | ATATTGTTGAACCAAACAGAAAGCT | | |
| | | TGAACCAAACAGAAAGCTCAAAATTAGGCACA | CAAAACTAGGCACAGAAAAGAGATA | | |
| | | GAAAAGAGATACAAACACAAATCTGAACAAAT | CAAACACAAATCTGAACAAATTGAC | | |
| | | GACCTTCTCCCTATAGCATAACTAATATCTCA | CTTCTCCCTATAGCATAACTAATAT | | |
| | | GAGATAAAAGTGGTCTTTATATACCAGGGCGA | CTAGAGATAAAAGTGGTCTTTATAT | | |
| | | AAGAGGTCTAAAAAGAGAGAAATAAAAAATAT | ACCAGGGCGAAAGAGGTCTAAAAAG | | |
| | | GGCATATTTCCTGTCATATGCAGAACCTATTG | AGAGAAATAAAAAATATGGCATATT | | |
| | | AGTCTTTTTGTTTGTTTCTTTCAATACAGCCT | TCCTGTCATATGCAGAACCTATATG | | |
| | | ATGTAGCTCTAGCTGTCCTAGAACTTACTTTG | AGTCTTTTTGTTTGTTTCTTTCAAT | | |
| | | TAGACCAGGCTTGCCGCGCCTGCAGCAGAAAT | ACAGCTATGTAGCTCTAGCTGTCCT | | |
| | | GGTTGAACTCCCGAGAGTGTCCTACACTTGGG | AGAACTTACTTTGTAGACCAGGCT\hat{ } | | |
| | | GAGAAGCAGCCAAGGGGTTGAAGGGCGAATTC | | | |
| IST000027 | MM0011B | GAATTCGCCCTTCGTGCTACACTATCAGTAGG | CATGTTGACAGTCCTAGAGCAAGAC | 163 | unknown |
| | | CATGTTGACAGTCCTAGAGCAAGACCTGAGGT | CTGAGGTAGAAGAATGGCCTTTAGA | | |
| | | AGAAGAATGGCCTTTAGAAAGTAGGGACTTCA | AAGTAGGGACTTCAGGTGCCTGAAA | | |
| | | GGTGCCTGAAAAATGGAGGGGTGCCAGGGCAA | AATGGAGGGGTGCCAGGGCAGATGT | | |
| | | TGTGGGGAGCCCTGTGAGGAGTGGGGTGTCCC | GGGGAGCCCTGTGAGGAGTGGGGTG | | |
| | | AGGGCAGATGTGGGGAGCCCTGTGAGGAGTGG | TCCAGGGCAGATGTGGGGAGCCCTG | | |
| | | GGTGCCGCGCCTGCAGCAGAAATGGTTGAACT | TGAGGAGTGGGG\hat{ } | | |
| | | CCCGAGAGTGTCCTACACTTAGGGGAGAAGAG | | | |
| | | CCAAGGGGTTGAAGGGCGAATTC | | | |
| IST000028 | MM0011C | GAATTCGCCCTTCGTGCTACACTATCAGTAGG | GATGTCAGAGCCGCAGGAATGCATA | 90 | unknown |
| | | GATGTCAGAGCCGCAGGAATGCATAATGGTCT | ATGGTCTTCTTTTTGTTTGTTTGTT | | |
| | | TCTTTTTGTTTGTTTGTTTTTTGAGACAGGGT | TTTTGAGACAGGGTTTCTCTGTGTA | | |
| | | TTCTCTGTGTAGCCCTGGCTGTCCTGTGCCGG | GCCCTGGCTGTCCTG\hat{ } | | |
| | | CCTGCAGCAGAAATGGTTGAACTCCCGAGAGT | | | |
| | | GTCCTACACTTAGGGGAGAAGCAGCCAAGGGG | | | |
| | | TTGAAGGGCGAATTC | | | |
| IST000029 | MM0011D | GAATTCGCCCTTGGTGCTACACTATCAGTAGG | CATGCAGGAGGCCCTCTGTCCTTAA | 40 | unknown |
| | | CATGCAGGAGGCCCTCTGTCCTTAACACAGAA | CACAGAATGTACAGG\hat{ } | | |
| | | TGTACAGGTGCCGCGCCTGCAGCAGAAATGGT | | | |
| | | TGAACTCCCGAGGGTGTCCTACACTTAGGGGG | | | |
| | | AAGCAGCCAAGGGGTTGTTTCCCACCAAGGAC | | | |
| | | GACCCGTCTGCGCACAAACGGATGAGCCCATC | | | |
| | | AAGGGCGAATTC | | | |
| IST000030 | MM0010D | GAATTCGCCCTTCGTGCTACACTATCAGTAGG | GATGCCCGATCAGTTGCTTCTAGAC | 48 | unknown |
| | | GATGCCCGATCAGTTGCTTCTAGACATAAAGA | ATAAAGAAAAATTCGTTGTTTTC\hat{ } | | |
| | | AAAATTCGTTGTTTTCTGCCGCGCCTGCAGCA | | | |
| | | GAAATGGTTGAACTCCCGAGAGTGTCCTACAT | | | |
| | | TAGGGGAGAAGCAGCCAAGGGGTTGAAGGGCG | | | |
| | | AATTC | | | |
| IST000031 | MM0012B | GAATTCGCCCTTCGTGCTACACTATCAGTAGGCATGTG | CATGTGAGTTCTTTTGTG | 187 | unknown |
| | | AGTTCTTTTGTGATTGGGTTACCTCACTCAGGATGATA | ATTGGGTTACCTCACTCA | | |
| | | CCCTCCAGATCCATCCATTTGCCTAGGAATGTCATAAA | GGATGATACCCTCCAGAT | | |
| | | TTCATTCTTTTTATAGCTGAGTAGTACTCCATTGTGTA | CCATCCATTTGCCTAGGA | | |
| | | AATGTACCACACTTTCTGTATCCATTCCTCTGTTGAGG | ATGTCATAAATTCATTCT | | |
| | | GGCATCTGGGTTCTTTCCAGCTTCTGGCTGCCGCGCCT | TTTTAATAGCTGAGTAGT | | |
| | | GCAGCAGAAATGGTTGAACTCCCGAGGTGTCCTACACT | ACTCCATTGTGTAAATGT | | |
| | | TAGGGGAGAAGCAGCCAAGGGGTAGAAGGGCGAATTC | ACACACTTTCTGTATCCA | | |
| | | | TTCCTCTGTTGAGGGGCA | | |
| | | | TCTGGGTTCTTTCCAGCT | | |
| | | | TCTGGC\hat{ } | | |
| IST000032 | MM0014D | GAATTCGCCCTTCGTGCTACACTATCAGTAGGGATGAT | GATGATG\hat{ } | 7 | too short |
| | | GTGCCGCGCCTGCAGCAGAAATGGTTGAACTCCCGAGA | | | |
| | | GTGTCCTACACTTAGGGGAGAAGCAGCCAAGGGGTTGA | | | |
| | | AGGGCGAATTC | | | |
| IST000033 | MM0018B | GAATTCGCCCTTCGTGCTACACTATCAGTAGGCATGGA | CATGGATCCTGACAACAG | 223 | unknown |
| | | TCCTGACAACAGGCTGAGTAAATGAAGCTGTTTATTCT | GCTGAGTAAATGAAGCTG | | |
| | | CAAGTGCTGAGGGCTGACCTGGAGCATACTGTCTAGAG | TTTATTCTCAAGTGCTGA | | |
| | | GGACCACTGCAGTTTCAAGTATGGATGGTGTTGAGCTT | GGGCTGACCTGGAGCATA | | |
| | | TGGGGGATGCTAGATGCATCTATAAATGTGTGCTGTTT | CTGTCTAGAGGGACCACT | | |
| | | ACTTTTTTTCATCTTGGTATATAATCTTTATTTGTACT | GCAGTCTTCAAGTATGGA | | |
| | | TTGTCAGTTTCATCTGTGTAGAAGATGCCGCGCCTGCA | TGGTGTTGAGCTTTGGGG | | |
| | | GCAGAAATGGTTGAACTCCCGAGAGTGTCCTACACTTA | GTGCTAGATGCATCTATA | | |
| | | GGGGAGAAGCAGCCAAGGGGTTGTTTCCCACCAAGGAC | AATGTGTGCTGTTTACTT | | |
| | | GACCCGTCTGCGCACAAACGGATGAGCCCATCAAGGGC | TTTTTCATCTTGGTATAT | | |
| | | GATTC | AATCTTTATTTGTACTTT | | |
| | | | GTCAGTTTCATCTGTGTA | | |
| | | | GAAGAT\hat{ } | | |
| IST000034 | MM0018C | GAATTCGCCCTTCGTGCTACACTATCAGTAGGCATGTT | CATGTTCCTTTGTCAAGG | 100 | unknown |
| | | CCTTTGTCAAGGTCTATGGACATTTCTGTCTTTTGGAA | TCTATGGACATTTCTGTC | | |
| | | TGTTCCTCCTTTAGATATCCTGGGGTTTTGTTGTTGTT | TTTTGGAATGTTCCTCCT | | |
| | | GTTTTGTTTTGAGCAGTTGCCGCGCCTGCAGCAGAAAT | TTAGATATCCTGGGGTTT | | |
| | | GGTTGAACTCCCGAGAGTGTCCTACACTTAGGGGAGAA | TGTTGTTGTTGTTTTGTT | | |
| | | GCAGCCAAGGGGTTGTTTCCCACCAAGGACGACCCGTC | TTGAGGCAGT\hat{ } | | |
| | | TGCGCACAAACGGATGAGCCCATCAAGGCGAATTC | | | |
| IST000035 | MM0019B | GAATTCGCCCTTCGTGCTACACTATCAGTAGGCATGAG | CATGAGCTGTCGATAGTG | 129 | unknown |
| | | CTGTCGATAGTGACCTGCAGTCAAGGAAATCTGAGGGC | ACCTGCAGTCAAGGAAAT | | |
| | | TTCCTAATTAACAGAGGAGCTCTAAATGAGAGTAACGC | CTGAGGGCTTCCTAATTA | | |
| | | GCTCCACAAACCCCTCACACTCGGTAAGTGTCACGGTG | ACAGAGGAGCTCTAAATG | | |
| | | CAGATAATTGCCGCGCCTGCAGCAGAAATGGTTGAACT | AGAGTAACGCGCTCCACA | | |
| | | CCCGAGAGTGTCCTACACTTAGGGGAGAAGCAGCCAAG | AACCCCCTCACACTCGGT | | |
| | | GGGTTGTTTCTCACCAAGGACGACCCTCTGCGCACAAA | AAGTGTCACGGTGCAGAT | | |
| | | CGGATGAGCCCATCAAGGGCGAATTC | AT\hat{ } | | |
| IST000036 | MM0021B | GAATTCGCCCTTCGTGCTACACTATCAGTAGGCATGAG | CATGAGCACTAAAGGCAG | 105 | unknown |
| | | CACTAAAGGCAGCTAACCAAAACAGAATCCCACAGACT | CTAACCAAAACAGAATCC | | |
| | | ACTCTGCAGCTTCTTAGGCTGGCTCGTTTTTCCAGTCT | CACAGACTACTCTGCAGC | | |
| | | AGCTATTGCCTTATTCCTCTTCTGCCGCGCCTACAACA | TTCTTAGGCTGGCTCGTT | | |
| | | GAAATGGTTGAACTCCCGAGAGTGTCCTACACTTAGGG | TTTCCAGTCTAGCTATTG | | |
| | | GAGAAGCAGCCAAGGGGTTGTTTCCCACCAAGGACGAC | CCTTATTTCCTCTTC\hat{ } | | |
| | | CCGTCTGCGCACAAACGGATGAGCCCTCAAGGGCGAAT | | | |
| | | TC | | | |
| IST000037 | MM0021C | GAATTCGCCCTTCGTGCTACACTATCAGTAGGCATGGA | CATGGATAGAGGATGGAA | 66 | unknown |
| | | TAGAGGATGGAAGTTGAAAACCTGCTATTAAGAATATA | GTTGAAAACCTGCTATTA | | |
| | | GCCCTGTCCATTAGTGAGAGTGTGCCGCGCCTGCAGCA | AGAATATAGCCCTGTCCA | | |
| | | GAAATGGTTGAACCCCGAGAGTGTCCTACACTTAGGGG | TTAGTGAGAGTG\hat{ } | | |
| | | AGAAGCAGCCAAGGGGTTGTTTCCCACCAAGGACGACC | | | |
| | | CGTCTGCGCACAAACGGATGAGCCCATCAAGGGCGAAT | | | |
| | | TC | | | |
| IST000038 | MM0024B | GAATTCGCCCTTCGTGCTACACTATCAGTAGGCATGAGGG | CATGAGGGGTACAAATT | 78 | unknown |
| | | GTACAAATTTGTTGTGCCTAAAAGCATTGAAGTGTAGGGA | TGTTGTGCCTAAAAGCA | | |
| | | AGACTGGGAAGTGGCACGGTCAGACTTAGGTGCCGCGCCT | TTGAAGTGTAGGGAAGA | | |
| | | GCAGCAGAATGGTTGAACTCCCGAGAGTGTCCTACACTTA | CTGGGAAGTGGCACGGT | | |
| | | GGGGAGAAACAGCCAAGGGGTTGTTTCCCACCAAGGACGA | CAGACTTAGG\hat{ } | | |
| | | CCCGTCTGCGCACAAACGGATGAGCCCATCAAGGGCGAAT | | | |
| | | TC | | | |
| IST000039 | MM0024C | GAATTCGCCCTTCGTGCTACACTATCAGTAGGCATGTCAC | CATGTCACACTGGGGAC | 46 | unknown |
| | | ACTGGGGACTCCCTCCGGGGAGGGAGCAGGCTGGGGGGTG | TCCCTCCGGGGAGGGAG | | |
| | | CCGCGCCTGCAGCAGAAATGGTTGAACTCCCGAGAGTGTC | CAGGCTGGGGGG\hat{ } | | |
| | | CTACACTAGGGGAGAAGCAGCCGAGGGGTTGTTTCCCACC | | | |
| | | AAGGACGACCCGTCTGCGCACAAACGGATGAGCCCATCAA | | | |
| | | GGGCGAATTC | | | |
| IST000040 | MM0022B | GAATTCGCCCTTCGTGCTACACTATCAGTAGGCATGGCTA | CATGGCTATCCTTTCCT | 99 | Wnt1 |
| | | TCCTTTCCTTTCATTGCTGCTACCTCCCCAGCTCTTGCAC | TTCATTGCTGCTACCTC | | |
| | | TAGGAGAGCTGGACTGTTGGTGTGCTGAGTCCCCATCAGG | CCCAGCTCTTGCACTAG | | |
| | | TGACACATTGTGCCGCGCCTGCAGCAGAAATGGTTGAACT | GAGAGCTGGACTGTTGG | | |
| | | CCCGAGAGTGTCCTACACTTAGGGGAGAAGCAGCCAAGGG | TGTGCTGAGTCCCCATC | | |
| | | GTTGTTTCCCACCAAGGACGACCCGTCTGCGCACAAACGG | AGGTGACACACTTG\hat{ } | | |
| | | ATGAGCCCATCAAGGCGAATTC | | | |
| IST000041 | MM0022C | GAATTCGCCCTTCGTGCTACACTATCAGTAGGCATGTCAG | CATGTCAGGTGGCTCAC | 85 | unknown |
| | | GTGGCTCACCTGCAGAGGATCTAATTAATGCCTCCACCCT | CTGCAGAGGATCTAATT | | |
| | | CCAGGAGTGCCTACCCTCACATAAACATATCCATATATGC | AATGCCTCCACCCTCCA | | |
| | | CGCGCCTCAGCAGAAATGGTTGAACTCCCGAGAGTGTCCT | GGAGTGCCTACCCTCAC | | |
| | | ACACTTAGGGGAGAAGCAGCCAAGGGGTTGTTTCCCACCA | ATAAACATATCCATATA\hat{ } | | |
| | | AGGACGACCCGTCTGCGCACAAACGGATGAGCCCATCAAG | | | |
| | | GGCGAATTC | | | |
| IST000042 | MM0022D | GAATTCGCCCTTCGTGCTACACTATCAGTAGGCATGTCTG | CATGTCTGCTGTCTAAA | 72 | unknown |
| | | CTGTCTAAACCTCCTAATCTATATATTTTGTTACAGATGT | CCTCCTAATCTATATAT | | |
| | | TCAAGATGACTACAGTCCACATATTGCCGCGCCTGCAGCA | TTTGTTACAGATGTTCA | | |
| | | GAAATGGTGAACTCCCGAGAGTGTCCTACACTTAGGGGAG | AGATGACTACAGTCCAC | | |
| | | AAGCAGCCAAGGGGTTGTTTCCCACCAAGGACGACCCGTC | ATAT\hat{ } | | |
| | | TGCGCACAAACGGATGAGCCCATCAAGGGCGAATTC | | | |
| IST000043 | MM0022F | GAATTCGCCCTTCGTGGTACACTATCAGTAGGCATGGTTC | CATGGTTCTGGGAA\hat{ } | 14 | too short |
| | | TGGGAATGCCGCGCCTGCAGCAGAAATGGTTGAACTCCCG | | | |
| | | AGAGTGTCCTACACTTAGGGGAGAAGCAGCCAAGGGGTTG | | | |
| | | TTTCCCACAAGGACGACCCGTCTGCGCACAAACGGATGAG | | | |
| | | CCCATCAAGGGCGAATTC | | | |
| IST000044 | MM0022G | GAATTCGCCCTTCGTGCTACACTATCAGTAGGCATGTGCC | CATG\hat{ } | 4 | too short |
| | | GCGCCTGCAGCAGAAATGGTTGAACTCCCGAGAGTGTCCT | | | |
| | | ACACTTAGGGGAGAAGCAGCCAAGGGGTTGTTTCCCACCA | | | |
| | | AGGACGACCGTCTGCGCACAAACGGATGAGCCCATCAAGG | | | |
| | | GCGAATTC | | | |
| IST000045 | MM0017B | GAATTCGCCCTTCGTGCTACACTATCAGTAGGCATGTGTC | CATGTGTCACTCAAAGG | 205 | unknown |
| | | ACTCAAAGGCTGCTGAGAATCAGGCTGTACCTGCATTCCT | CTGCTGAGAATCAGGCT | | |
| | | AAGCCATCCACAGCCATCCTGACCCACAGCAAATGCTGGC | GTACCTGCATTCCTAAG | | |
| | | AGTCGCCCACAGCTGGACTCCGTTCCTCCCTCCACTCCTA | CCATCCACAGCCATCCT | | |
| | | TAGCCGAGGCTATCCACACAGGCTATTTCAGTGCCCTAAG | GACCCACAGCAAATGCT | | |
| | | CCTTGCTACCCTTATGTATACATTGAGGACAATGACTGCC | GGCAGTCGCCCCACAGC | | |
| | | GCGCCTGCAGCAGAATGGTTGAACTCCCGAGAGTGTCCTA | TGGACTCCGTTCCTCCC | | |
| | | CACTAAGGGGAGAAGCAGCCAAGGGGTTGTTTCCCACCAA | TCCACTCCATAGCCGAG | | |
| | | GGACGACCCGTCTGCGCACAAACGGATGAGCCCATCAAGG | GCTATCCACACAGGCTA | | |
| | | GCGAATTC | TTTCAGTGCCCTAAGCC | | |
| | | | TTGCTACCCTTATGTAT | | |
| | | | ACATTGAGGACAATGAC\hat{ } | | |
| IST000046 | MM0017D | GAATTCGCCCTTCGTGCTACACTATCAGTAGGCATGGAA | CATGGAAATA\hat{ } | 10 | too short |
| | | ATATGCCCCGCCTGCAGCAGAAATGGTTGAACTCCCGAG | | | |
| | | AGTGTCCTACACTTAGGGGAGAAGCAGCCAAGGGGTTGT | | | |
| | | TTCCCACCAAGACGACCCGTCTGCGCACAAACGGATGAG | | | |
| | | CCCATCAAGGGCGAA~TTC | | | |
| IST000047 | MM0017E | GAATTCGCCCTTCGTGCTACACTATCAGTAGGCATGCCG | CA\hat{ } | 2 | too short |
| | | CGCCTGCAGCAGAAATGGTTGAACTCCCGAGAGTGTCCT | | | |
| | | ACACTTAGGGGAGAAGCAGCCAAGGGGTTGTTTCCCACC | | | |
| | | AAGGACGACCGTCTGCGCACAAACGGATGAGCCCATCAA | | | |
| | | GGGCGAATTC | | | |
| IST000048 | MM0020B | GAATTCGCCCTTCGTGCTACACTATCAGTAGGCATGGAG | CATGGAGAAACCTGGGCT | 126 | unknown |
| | | AAACCTGGGCTTATTCAAGCAGTTTCCTTTGTTTACCCT | TATTCAAGCAGTTTCCTT | | |
| | | GCCCAGGGTTGCCAGTGAAGGGGCTCCTCCATCACTAAC | TGTTTACCCTGCCCAGGG | | |
| | | TAAAGGTCTTTCCTATGCTGGTTCCTCTCCACCCCACCA | TTGCCAGTGAAGGGGCTC | | |
| | | TTGCCGCGCCTGCAGCAGAAATGGTTGAACTCCCGAGAG | CTCCATCACTAACTAAAG | | |
| | | TGTCCTACACTTAGGGGAGAAGCAGCCAAGGGGTTGTTT | GTCTTATCCTATGCTGGT | | |
| | | CCCACCAAGGACGACCCGTCGCGCACAAACGGATGAGCC | TCCTCTCCACCCCACCAT\hat{ } | | |
| | | CATCAAGGGCGAATTC | | | |
| IST000049 | MM0020D | GAATTCGCCCTTCGTGCTACACTATCAGTAGGCATGGGT | CATGGGTGTG\hat{ } | 10 | too short |
| | | GTGTGCCGCGCCTGCAGCAGAAATGGCTGAACTCCCGAG | | | |
| | | AGTGTCCTACACTTAGGGGAGAAGCAGCCAAGGGGTTGT | | | |
| | | TTCCCACCAAGACGACCCGTCTGCGCACAAACGGATGAG | | | |
| | | CCCATCAAGGGCGAATTC | | | |
| IST000050 | MM0023B | GAATTCGCCCTTCGTGCTACACTATCAGTAGGCATGTCC | CATGTCCTCCACAGCCGA | 221 | Fgf3 |
| | | TCCACAGCCGAGCACCCTCAACTGTCTCCCAGGGCCTCT | GCACCCTCAACTGTCTCC | | |
| | | GTTCTAGCCAGGGTCTGCAGGGTCTCTGCCCCACGCCTA | CAGGGCCTCTGTTCTAGC | | |
| | | GCCCCTGAGAATCTTAAGCGGTCTGAAAACTACGCCACT | CAGGGTCTGCAGGGTCTC | | |
| | | GAACTGCTAAAACCCTGGAGTCACTGATGGAACCTGCTT | TGCCCCACGCCTAGCCCC | | |
| | | TACAGGGGACAGCCCTGTGTCTCGAAGACCAACCTAAGC | TGAGAAATCTTAAGCGGT | | |
| | | TCCTAAGATGTCACAAATTGCGCGCCTGCAGCAGAAATG | CTGAAAACTACGCCACTG | | |
| | | GTTGAACTCCCGAGAGTGTCCTACACTTAGGGGAGAAGC | ACTGCTAAAACCCTGGAG | | |
| | | AGCCAAGGGGTTGTTTCCCACCAAGGACGACCCGTCTGC | TCACTGATGGAACCTGCT | | |
| | | GCACAAACGGATGAGCCCATCAAGGGCGAATC | TTACAGGGGACAGCCCTG | | |
| | | | TGTCTCGAAGACCAACCT | | |
| | | | AAGGTCCTAAGATGTCAC | | |
| | | | AAAT\hat{ } | | |
| IST000051 | MM0023C | GAATTCGCCCTTCGTGCTACACTATCAGTAGGCATGACG | CATGACGACTTGAAAAAT | 39 | unknown |
| | | ACTTGAAAAATGACGAAATCACTAAAAAACAATGCCGCG | GACGAAATCACTAAAAAA | | |
| | | CCTGCAGCAGAAATGGTTGAACTCCCGAGAGTGTCCTAC | CAA\hat{ } | | |
| | | ACTTAGGGGAAAGCAGCCAAGGGGTTGTTTCCCACCAAG | | | |
| | | GACGACCCGTCTGCGCACAAACGGATGAGCCCATCAAGG | | | |
| | | GCGAATTC | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid molecule as shown in Table 1.
